(19) United States
(12) Patent Application Publication
(10) Pub. No.: US 2011/0184322 A1
(43) Pub. Date: Jul. 28, 2011

(54) METHOD AND DEVICE FOR TREATMENT OF KELOIDS AND HYPERTROPHIC SCARS USING FOCUSED ULTRASOUND

(75) Inventors: Solli Brawer, Kiryat Ono (IL); Haim Azhari, Doar-Na Misgav (IL); Liat Tsoref, Tel Aviv (IL)

(73) Assignee: SLENDER MEDICAL LTD., Herzliya (IL)

(21) Appl. No.: 13/010,555

(22) Filed: Jan. 20, 2011

Related U.S. Application Data
(60) Provisional application No. 61/297,286, filed on Jan. 22, 2010.

(51) Int. Cl. A61N 7/00

(52) U.S. Cl. 601/3

(57) ABSTRACT

A method for treating a scar on a skin surface of a subject is provided. A subject is identified as having a skin surface with scar tissue. In response to identifying the subject as having the scar tissue, a housing comprising at least one acoustic transducer is placed on the skin surface with the scar tissue and the acoustic transducer is activated to apply high intensity focused ultrasound energy to a portion of the scar tissue. Other embodiments are also described.

### CROSS-REFERENCES TO RELATED APPLICATIONS

The present application claims the priority of U.S. Provisional Application 61/297,286 to Brawer, entitled, “Method and device for treatment of keloids and hypertrophic scars using focused ultrasound,” filed Jan. 22, 2010, which is incorporated herein by reference.

### FIELD OF THE APPLICATION

The present invention relates generally to treatment of scars and particularly to methods and apparatus for treatment of hypertrophic scars and keloids, by application of focused ultrasound energy thereto.

### BACKGROUND OF THE APPLICATION

Wound healing in certain individuals may lead to the development of hypertrophic scars and/or keloids. Hypertrophic scars typically take the form of a raised scar on the skin, and occur due to excessive production of collagen during a wound healing process. Hypertrophic scars typically do not grow beyond the boundaries of the original wound area.

In contrast, keloids, or keloidal scars, expand beyond the original wound site and may grow into a firm lump that is many times larger than the original scar. Keloids are typically fibrotic growths that contain a collection of atypical fibroblasts and an increased abundance of extracellular matrix components, especially collagen.

Although commonly benign, hypertrophic scars and keloids often cause discomfort, pain, pruritus, physical disfigurement and impaired quality of life.

Current approaches for treatment of hypertrophic scars and/or keloids include silicone devices for applying local pressure, injection of various materials (e.g. steroids, verapamil, 5-FU, etc.), freezing the tissue with liquid nitrogen, laser ablation, surgical removal and ionizing radiation.

### SUMMARY OF APPLICATIONS

In some applications of the present invention, a method and apparatus for treating scar tissue on a skin surface of a subject are provided. Typically, a subject is identified as having a skin surface with scar tissue. For some applications, the scar tissue includes a hypertrophic scar and/or a keloid scar. In response to identifying the subject as having the scar, a housing comprising at least one acoustic transducer, e.g., an ultrasound transducer, is placed on the skin surface with the scar tissue. The ultrasound transducer is activated to apply high intensity focused ultrasound (HIFU) energy to the scar tissue. Application of high intensity focused ultrasound energy to the scar tissue typically leads to elevation of the temperature within the scar tissue.

As described hereinabove, the hypertrophic and/or keloid scar tissue contains an accumulation of collagen. Elevation of the temperature within the scar tissue to a critical temperature typically results in heat-induced changes in the collagen, e.g., denaturing of collagen molecules typically when the temperature is raised to or above 60 C. Thermal denaturing of collagen generally occurs by the dissociation of heat-sensitive bonds of the collagen molecule and may result in denaturing of collagen into gelatin as described in an article by Harel A et al., entitled, “Magnetization transfer based contrast for imaging denatured collagen” J Magn Reson Imaging. 2008 May; 27(5):1155-63, which is incorporated herein by reference. As provided by some applications of the present invention, the focused ultrasound energy that is applied to the scar tissue is capable of elevating the temperature within the scar tissue to a temperature that is sufficient to cause denaturing of collagen within the scar tissue. For some applications, heating of the scar tissue results in denaturing the collagen into gelatin. The gelatin is typically softer than collagen, and thus may relieve discomfort, pain, pruritus and/or physical disfigurement that are associated with the scar tissue. Additionally, softer, gelatin containing scar tissue, may be easily removed.

For some applications, following application of high intensity focused ultrasound energy to the scar tissue, a suction device is applied to remove treated scar tissue from the skin. For applications in which elevation of the temperature within the scar tissue leads to collagen denaturating to gelatin, the gelatin is removed from the scar tissue.

Additionally or alternatively, elevation of the temperature within the scar tissue by use of high intensity focused ultrasound energy results in destruction of collagen-producing fibroblast cells within the scar tissue, thereby reducing further production of collagen.

There is therefore provided, in accordance with some applications of the present invention, a method for treating a scar on a skin surface of a subject, the method including:
identifying a subject as having a skin surface with scar tissue;

in response to identifying the subject as having the scar tissue, placing a housing including at least one acoustic transducer on the skin surface with the scar tissue; and

activating the acoustic transducer to apply high intensity focused ultrasound energy to a portion of the scar tissue.

For some applications, the scar tissue includes a hypertrophic scar, and applying high intensity focused ultrasound energy to the scar tissue includes applying high intensity focused ultrasound energy to the hypertrophic scar.

For some applications, the scar tissue includes a keloid scar, and applying high intensity focused ultrasound energy to the scar tissue includes applying high intensity focused ultrasound energy to the keloid scar.

For some applications, activating the acoustic transducer to apply high intensity focused ultrasound energy to the scar tissue includes elevating the temperature of the scar tissue.

For some applications, elevating the temperature of the scar tissue includes elevating the temperature to at least 57 C.

For some applications, activating the acoustic transducer to apply high intensity focused ultrasound energy to the scar tissue includes inducing cavitation of the scar tissue.

For some applications, the housing includes a cooling fluid and the method further includes cooling the skin surface with the cooling fluid.

For some applications, the cooling fluid includes liquid nitrogen, and cooling includes cooling the skin surface with the liquid nitrogen.

For some applications the method includes, suctioning treated tissue following application of high intensity focused ultrasound energy to the scar tissue.

For some applications the method includes, repositioning the acoustic transducer following activation of the acoustic transducer, and subsequently to the repositioning, activating the acoustic transducer to apply high intensity focused ultrasound energy to another portion of the scar tissue.

There is further provided, in accordance with some applications of the present invention, a method for treating a scar on a skin surface of a subject, the method including:

identifying a subject as having a skin surface with scar tissue;

in response to identifying the subject as having the scar tissue, clamping a portion of the scar tissue using at least one acoustic transducer; and

activating the at least one acoustic transducer to apply high intensity focused ultrasound energy to the portion of the scar tissue.

For some applications the method includes, reflecting the transmitted energy from the acoustic transducer toward the scar tissue with an acoustic reflector.

For some applications, the scar tissue includes a hypertrophic scar, and applying high intensity focused ultrasound energy to the scar tissue includes applying high intensity focused ultrasound energy to the hypertrophic scar.

For some applications, the scar tissue includes a keloid scar, and applying high intensity focused ultrasound energy to the scar tissue includes applying high intensity focused ultrasound energy to the keloid scar.

For some applications, activating the acoustic transducer to apply high intensity focused ultrasound energy to the scar tissue includes elevating the temperature of the scar tissue.

For some applications, elevating the temperature of the scar tissue, includes elevating the temperature to at least 57 C.

For some applications, activating the acoustic transducer to apply high intensity focused ultrasound energy to the scar tissue includes inducing cavitation of the scar tissue.

There is still further provided, in accordance with some applications of the present invention, apparatus including:

a housing adapted for placement on a skin surface with scar tissue;

at least one acoustic transducer coupled to the housing and configured to apply high intensity focused ultrasound energy to portion of the scar tissue; and

a liquid nitrogen cooling fluid surrounding the acoustic transducer.

There is yet additionally provided, in accordance with some applications of the present invention, apparatus including:

a housing adapted for placement on a skin surface with scar tissue;

at least one acoustic transducer coupled to the housing and configured to apply high intensity focused ultrasound energy to a portion of the scar tissue for treatment of the scar tissue; and

a suction device configured to remove the treated scar tissue following treatment.

For some applications the apparatus includes, a sensing element configured to generate a signal that activates the suction device.

For some applications, the sensing element includes a temperature sensor configured to sense a temperature of the scar tissue, and, in response, activate the suction device.

For some applications, the scar tissue includes a hypertrophic scar, and the at least one acoustic transducer is configured to apply high intensity focused ultrasound energy to the hypertrophic scar.

For some applications, the scar tissue includes a keloid scar, and the at least one acoustic transducer is configured to apply high intensity focused ultrasound energy to the keloid scar.

For some applications, the at least one acoustic transducer is configured to apply high intensity focused ultrasound energy to the scar tissue to elevate the temperature of the scar tissue.

For some applications, the at least one acoustic transducer is configured to elevate the temperature of the scar tissue to at least 57 C.

For some applications, the housing includes a cooling fluid configured to cool the skin surface.

For some applications, the cooling fluid includes liquid nitrogen.

For some applications, the housing includes a base portion that is transparent to ultrasound waves and has high thermal conductivity.

The present invention will be more fully understood from the following detailed description of applications thereof, taken together with the drawings, in which:

### BRIEF DESCRIPTION OF THE DRAWINGS

FIG. 1 is a schematic illustration of apparatus and a method for treating scar tissue on a skin surface of a subject, in accordance with some applications of the present invention;

FIG. 2 is a schematic illustration of an ultrasound transducer for treatment of scar tissue, and a suction device operable in combination with the ultrasound transducer, in accordance with some applications of the present invention;

FIG. 3 is a schematic illustration of apparatus for treatment of scar tissue, in accordance with some applications of the present invention; and

FIGS. 4A-B are schematic illustrations of configurations of the apparatus for treating scar tissue, positioned in contact with the scar tissue, in accordance with some applications of the present invention.

### DETAILED DESCRIPTION OF APPLICATIONS

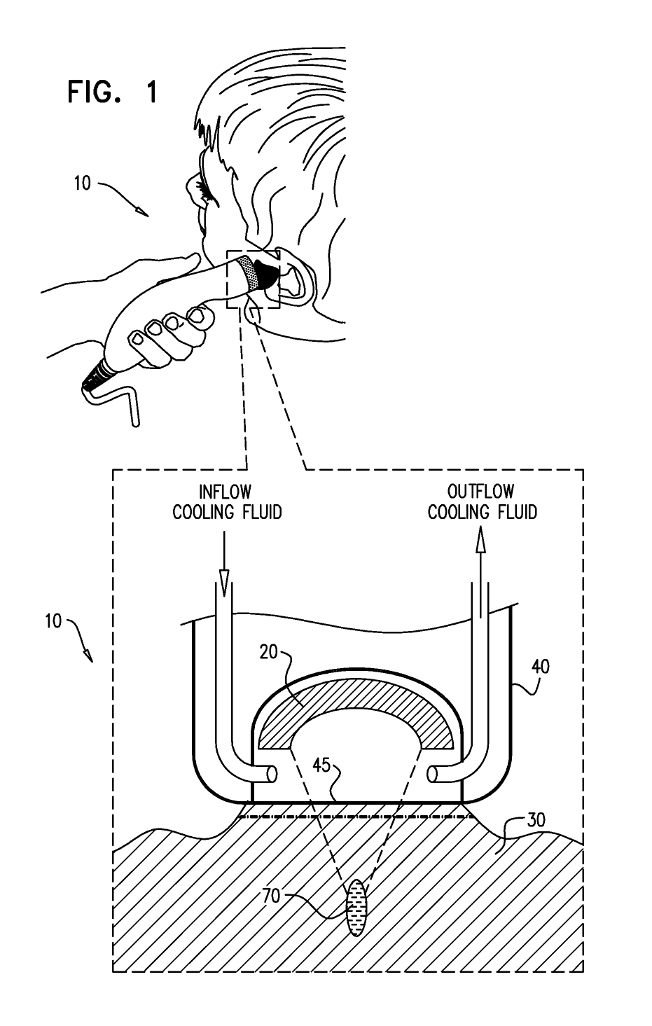

FIG. 1 is a schematic illustration of an apparatus and method for treating scar tissue on a skin surface of a subject by application of focused ultrasound energy thereto, in accordance with some applications of the present invention. For some applications, the scar tissue includes a hypertrophic scar and/or a keloid scar.

For some applications, apparatus 10 is provided for the treatment of scar tissue 30, e.g., a hypertrophic scar and/or a keloid scar. Typically, apparatus 10 comprises a housing 40 comprising at least one ultrasound transducer 20. Apparatus 10 is configured for placement on scar tissue on a skin surface of a subject. Ultrasound transducer 20 is configured to apply high intensity focused ultrasound energy to the scar tissue for treatment, e.g., removal and/or reduction, of the scar tissue.

Apparatus 10 is typically used with a suitable acoustic coupling fluid. Additionally, housing 40 comprises a base portion 45, which is typically transparent to ultrasound waves yet has high thermal conductivity. For some applications, base portion 45 comprises a thin membrane composed of a polyether film. Apparatus 10 is typically configured to focus energy transmission to a particular area of the scar tissue in order to enable treatment at a desired focal zone 70. Typically, focal zone 70 is located several millimeters under the base portion, for example 1-15 mm from the base portion.

For some applications, apparatus 10 applies high intensity focused ultrasound to scar tissue 30 at a frequency of 0.5-10 MHz, e.g., 2-5 MHz. Application of high intensity focused ultrasound energy to scar tissue 30 typically leads to a rapid rise in temperature within the scar tissue, typically to a temperature that is higher than 57 C, e.g. 58-80 C, e.g. 58-70 C. The treated scar tissue is thus ablated as a result of the rise in temperature, and is typically naturally removed by the body during the weeks following the treatment. For some applications, apparatus 10 is configured to apply lower frequencies, e.g. frequencies of 50-500 kHz. Application of energy in such frequencies, typically in combination with pulse sequences having a duty cycle which is less than 20% may create an effect of cavitation in the scar tissue and consequently, lead to destruction of the scar tissue. It is to be noted that techniques described with reference to creating cavitation in PCT Publication WO 2000-53263 to Rosenschein, may be practiced in combination with techniques described herein.

Additionally or alternatively, elevation of the temperature within the scar tissue results in heat-induced denaturing of collagen in the scar tissue. For some applications, heating of the scar tissue results in denaturing of the collagen into gelatin. The gelatin is typically softer than collagen, and thus may relieve discomfort, pain, pruritus and/or physical disfigurement that are associated with the scar tissue. Additionally, softer, gelatin-containing scar tissue may be easily removed. Further additionally, elevation of the temperature within the scar tissue by use of high intensity focused ultrasound energy may result in destruction of collagen-producing fibroblast cells within the scar tissue, thereby reducing further production of collagen.

For some applications, the ultrasound energy emitted from transducer 20 is focused such that focal zone 70 is disposed beneath the surface of the skin containing the scar, so as to reduce or avoid damage to the surface of the skin. Ultrasound transducer 20 is typically surrounded by a cooling fluid that flows through housing 40 in proximity to the skin surface and removes excess heat from transducer 20 and/or from the surface of the skin. Thus, apparatus 10 is configured to simultaneously heat a desired area in scar tissue 30, while preventing heating of other areas of the scar tissue as well as adjacent skin surface and tissue areas. Typically, the area above the dashed line in scar tissue 30, shown in FIG. 1, is generally not heated as a result of the high intensity focused ultrasound applied to scar tissue 30.

For some applications, the cooling fluid comprises a very low temperature cooling fluid, such as liquid nitrogen, which is kept close to the freezing temperature of the fluid (e.g., 63-70 K), in order to maintain the skin surface at a very low temperature. For these applications, housing 40 typically comprises a base portion 45, which is typically transparent to ultrasound waves yet has high thermal conductivity.

For some applications, the skin surface and adjacent tissue may be frozen, e.g., by being brought to a temperature of 0-10 C. Lowering the temperature of the skin surface and tissue in the vicinity of the heated focal zone may reduce the occurrence of damage to the skin, e.g., skin burns, as a result of thermal treatment of the skin containing scar tissue 30. Additionally or alternately, if freezing is achieved and sustained for adequate time, non-invasive tissue destruction of the skin and the tissue layers proximal to it is achieved. This may lead to skin rejuvenation and better cosmetic effect.

It is to be noted that for some applications, apparatus 10 does not use a cooling fluid.

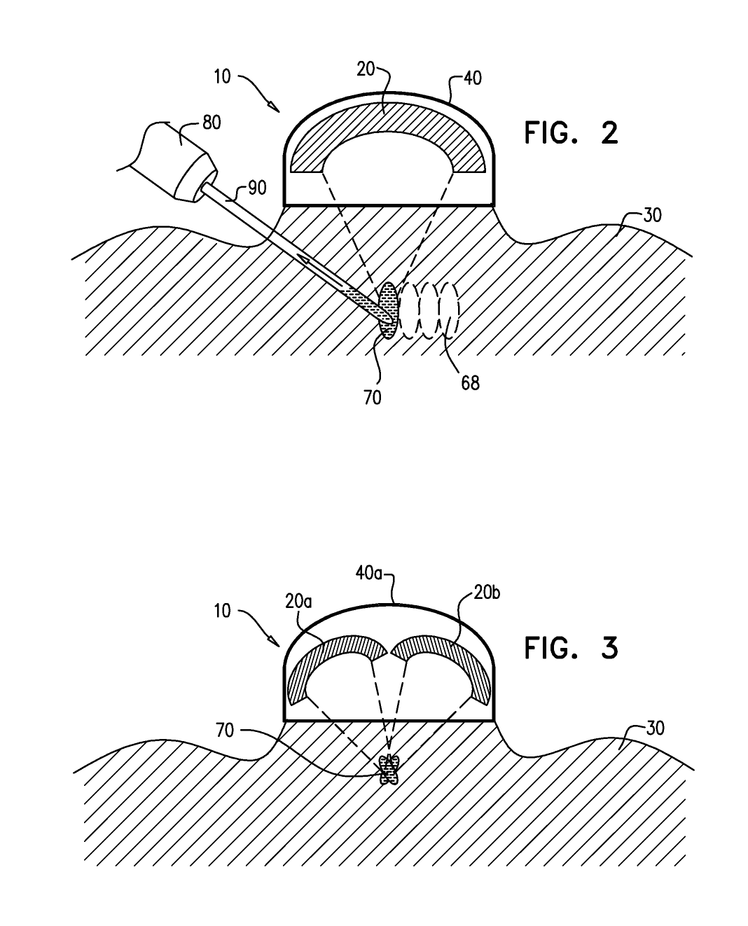

Reference is made to FIG. 2 which is a schematic illustration of an ultrasound transducer 20 for treatment of scar tissue 30, and a suction device 80 operable in combination with the ultrasound transducer, in accordance with some applications of the present invention. As described hereinabove with reference to FIG. 1, apparatus 10 applies high intensity focused ultrasound energy to scar tissue 30, leading to a rise in temperature within the scar tissue, typically to a temperature that is higher than 57 C, e.g. 58-80 C, e.g. 58-70 C. This thermal treatment of tissue 30 typically results in destructions of cells, e.g., fibroblast cells, and other components of the scar tissue. Additionally or alternatively, the elevation of temperature in scar tissue 30 leads to thermally-induced changes in collagen, e.g., denaturing of collagen into gelatin. Therefore, thermal treatment of the scar tissue yields a softer, more fluid, tissue which can be removed by suction device 80. Additionally or alternatively, cell debris may be removed following treatment of the scar tissue. For some applications, subsequently to or simultaneously with application of the ultrasound energy to the scar tissue, suction device 80 is operated in order to remove the treated tissue.

Typically, suction device 80 comprises a needle 90, at a distal end thereof. Needle 90 is typically inserted into scar tissue 30, in a vicinity of focal zone 70. Suction device 80 is operated to remove the treated scar tissue through needle 90. Needle 90 may be inserted into scar tissue 30 at any stage prior to, during, or following treatment of scar tissue 30.

The apparatus may comprise a sensing element configured to generate a signal that activates suction device 80. For some applications, needle 90 comprises the sensing element. Typically, the sensing element is configured to sense a temperature of the scar tissue and generate a signal for activation of suction device 80 when the scar tissue reaches a temperature that is sufficient for converting the scar tissue into a softer, more fluid tissue, e.g., 60 C. For such applications, needle 90 is inserted into scar tissue 30 prior to or during treatment of the scar tissue. Application of the focused ultrasound to scar tissue 30 causes a rise in temperature within the scar tissue triggering activation of suction device 80 when the scar tissue reaches a desired temperature.

Additionally or alternatively, ultrasound transducer 20 is configured, in addition to applying high intensity treatment energy, to transmit short low intensity ultrasound pulses and receive their echo. The received echo signals may be analyzed to detect a change in the fluidity of the scar tissue that typically occurs as a result of thermal treatment to the tissue, and in response, to terminate application of the high intensity treatment energy and/or to activate suction device 80.

Reference is made to FIG. 3, which is a schematic illustration of apparatus for treatment of scar tissue 30, in accordance with some applications of the present invention. For some applications, housing 40 as described with reference to FIGS. 1-2 is configured to house more than one ultrasound transducer 20. FIG. 3 shows housing 40a comprising more than one transducer 20. FIG. 3 shows housing 40a comprising two ultrasound transducers, 20a and 20b, by way of illustration and not limitation. Housing 40a may comprise any suitable number of ultrasound transducers 20. Typically, transducers 20a and 20b are confocal transducers, having substantially the same focal zone 70. Transducers 20a and 20b are configured to simultaneously or alternately apply high intensity focused energy for treatment, by heating and/or inducing cavitation of scar tissue 30, as described herein.

For some applications, e.g., at predetermined time intervals, one of transducers 20a or 20b may function as a receiver for detecting scattered waves from the treatment area, in the vicinity of focal zone 70, while the other transducer continues to apply treatment energy. The detected waves may be analyzed and used for monitoring changes in the scar tissue as a result of the treatment process, by measuring parameters such as scatter intensity, attenuation, speed of sound, non-linear parameters, sub harmonics and second harmonic reflections, thus allowing non-invasive monitoring of the treatment process.

Reference is made to FIGS. 1-3. For some applications, any transducer 20 (i.e., transducer 20, 20a and 20b) may be steered, rotated, or moved over the skin, in order to change the location of focal zone 70. Such movements of transducer 20 typically allow treatment of a larger area of scar tissue 30 (see dashed focal zones 70 in FIG. 2). For some applications, steering of transducer 20 is performed by a robotic mechanism that is coupled to transducer 20. Any other suitable steering mechanism may be used.

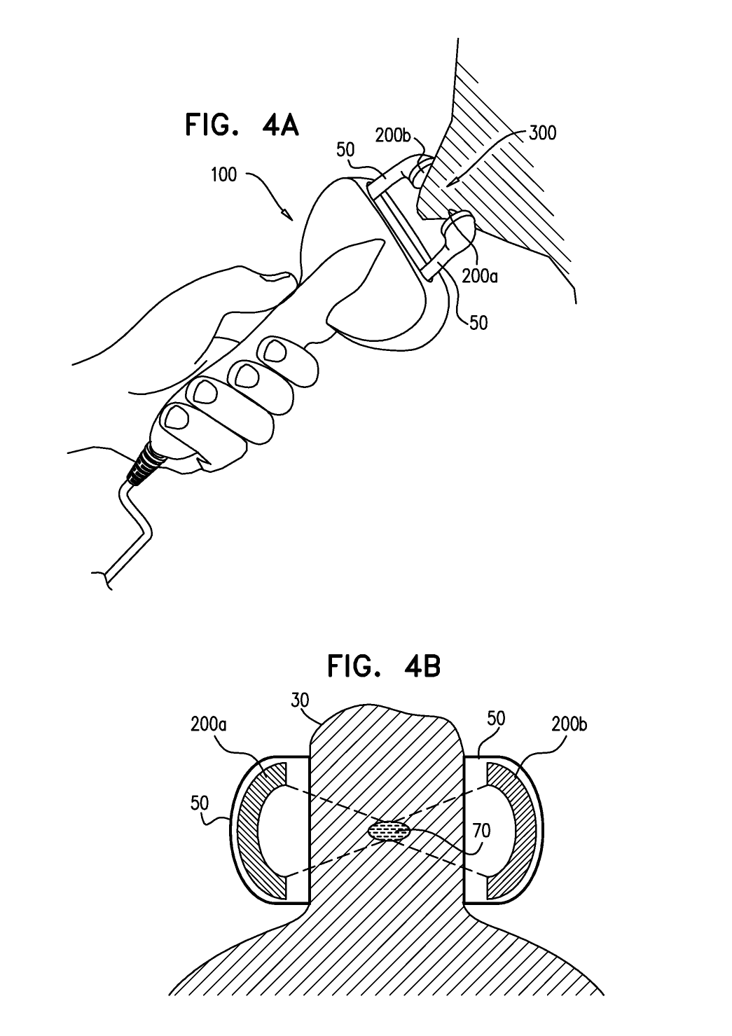

Reference is made to FIGS. 4A-B, which are schematic illustrations of configurations of apparatus 100 for treating scar tissue, positioned in contact with the scar tissue, in accordance with some applications of the present invention. Some or all apparatus and methods described hereinabove with reference to FIGS. 1-3 are typically utilized with the embodiments shown in FIGS. 4A-B, except with differences as described below.

For some applications, apparatus 100 comprises a housing 400, which typically comprises first and second clamping elements 50, which are configured to clamp a portion of scar tissue 30 therebetween. Typically, confocal ultrasound transducers 200a and 200b are coupled to clamping elements 50 and are configured to transmit focused high intensity ultrasound energy for thermal treatment of the scar tissue, particularly in focal zone 70. Transducers 200a and 200b are typically activated alternately or simultaneously to heat the same tissue region. For some applications, apparatus 100 comprises a single ultrasound transducer coupled to one of clamping elements 50. Additionally, the other clamping element may be coupled to an acoustic reflector, e.g., a mirror with a concave or flat surface, configured to reflect the energy transmitted from the ultrasound transducer toward focal zone 70. For some applications, techniques described with reference to an acoustic reflector in PCT publication WO 2010/029556 to Azhari et al., may be practiced in combination with techniques described herein.

Typically, transducers 200a and 200b are configured to transmit energy through the scar tissue, such that at least a portion of the transmitted energy reaches the other transducer by through transmission through the scar tissue. For some applications, e.g., at predetermined time intervals, one of transducers may function as a receiver for detecting scattered waves from the treatment area, in the vicinity of focal zone 70, while the other transducer continues to apply treatment energy. The detected waves may be analyzed and used for monitoring changes in the scar tissue as a result of the treatment process, by measuring parameters such as scatter intensity, attenuation, speed of sound, non-linear parameters, sub harmonics and second harmonic reflections, thus allowing non-invasive monitoring of the treatment process.

The following is a proposed, non-limiting, treatment procedure for application, as appropriate, with the apparatus and method described herein. Prior to treatment, the scar tissue is cleaned using 70% isopropyl alcohol wipe and shaved if required. The area designated for treatment is typically dotted with a pen at 2-3 mm intervals, and an ultrasonic gel is applied to the designated area. For applications in which the clamping elements are used, the physician typically clamps the skin containing the scar tissue such that a portion of the tissue is clamped between the ultrasound transducers and against a marked dot. The ultrasound transducers are then activated to apply energy to the tissue as described hereinabove. Typically, the duration of energy transmission from the transducers is between 0.1-4 seconds. Following each treatment, the transducers may be repositioned for subsequent transmission until the entire designated area has been treated. Typically, the number of treatments varies, as appropriate for the size of the scar. Total treatment duration may be, for example, 30 minutes.

Reference is made to FIG. 1-4B. Treatments using the apparatus and methods described herein may include, as appropriate, causing heating, tissue damage, thermal ablation, mechanical irritation, cell structure alteration, and/or a cavitation effect. Typically, the treatment system comprises circuitry for configuring the applied energy as high intensity focused ultrasound (HIFU), using techniques known in the art.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A method for treating a scar on a skin surface of a subject, the method comprising:
identifying a subject as having a skin surface with scar tissue;
in response to identifying the subject as having the scar tissue, placing a housing comprising at least one acoustic transducer on the skin surface with the scar tissue; and
activating the acoustic transducer to apply high intensity focused ultrasound energy to a portion of the scar tissue.

2. The method according to claim 1, wherein the scar tissue includes a hypertrophic scar, and wherein applying high intensity focused ultrasound energy to the scar tissue comprises applying high intensity focused ultrasound energy to the hypertrophic scar.

3. The method according to claim 1, wherein the scar tissue includes a keloid scar, and wherein applying high intensity focused ultrasound energy to the scar tissue comprises applying high intensity focused ultrasound energy to the keloid scar.

4. The method according to claim 1, wherein activating the acoustic transducer to apply high intensity focused ultrasound energy to the scar tissue comprises elevating the temperature of the scar tissue.

5. The method according to claim 4, wherein elevating the temperature of the scar tissue comprises elevating the temperature to at least 57 C.

6. The method according to claim 1, wherein activating the acoustic transducer to apply high intensity focused ultrasound energy to the scar tissue comprises inducing cavitation of the scar tissue.

7. The method according to claim 1, wherein the housing includes a cooling fluid and wherein the method further comprises cooling the skin surface with the cooling fluid.

8. The method according to claim 7, wherein the cooling fluid includes liquid nitrogen, and wherein cooling comprises cooling the skin surface with the liquid nitrogen.

9. The method according to claim 1, further comprising suctioning treated tissue following application of high intensity focused ultrasound energy to the scar tissue.

10. The method according to claim 1, further comprising repositioning the acoustic transducer following activation of the acoustic transducer, and subsequently to the repositioning, activating the acoustic transducer to apply high intensity focused ultrasound energy to another portion of the scar tissue.

11. A method for treating a scar on a skin surface of a subject, the method comprising:
identifying a subject as having a skin surface with scar tissue;
in response to identifying the subject as having the scar tissue, clamping a portion of the scar tissue using at least one acoustic transducer; and
activating the at least one acoustic transducer to apply high intensity focused ultrasound energy to the portion of the scar tissue.

12. The method according to claim 11, further comprising reflecting the transmitted energy from the acoustic transducer toward the scar tissue with an acoustic reflector.

13. The method according to claim 11, wherein the scar tissue includes a hypertrophic scar, and wherein applying high intensity focused ultrasound energy to the scar tissue comprises applying high intensity focused ultrasound energy to the hypertrophic scar.

14. The method according to claim 11, wherein the scar tissue includes a keloid scar, and wherein applying high intensity focused ultrasound energy to the scar tissue comprises applying high intensity focused ultrasound energy to the keloid scar.

15. The method according to claim 11, wherein activating the acoustic transducer to apply high intensity focused ultrasound energy to the scar tissue comprises elevating the temperature of the scar tissue.

16. The method according to claim 15, wherein elevating the temperature of the scar tissue, comprises elevating the temperature to at least 57 C.

17. The method according to claim 11, wherein activating the acoustic transducer to apply high intensity focused ultrasound energy to the scar tissue comprises inducing cavitation of the scar tissue.

18-28. (canceled)
